(19) United States
(12) Patent Application Publication
(10) Pub. No.: US 2022/0330802 A1
(43) Pub. Date: Oct. 20, 2022

(54) ENDOSCOPE AND METHOD FOR MANUFACTURING ENDOSCOPE

(71) Applicant: HOYA CORPORATION, Tokyo (JP)

(72) Inventor: Toshiki WATANABE, Tokyo (JP)

(73) Assignee: HOYA CORPORATION, Tokyo (JP)

(21) Appl. No.: 17/642,395

(22) PCT Filed: Feb. 10, 2021
(86) PCT No.: PCT/JP2021/004941

§ 371 Date: Mar. 11, 2022

(30) Foreign Application Priority Data
Mar. 23, 2020 (JP) 2020-051567

(51) Int. Cl. A61B 1/12; A61B 1/06; A61B 1/00

(52) U.S. Cl. CPC A61B 1/126; A61B 1/0684; A61B 1/00096; A61B 1/00094

(57) ABSTRACT

Provided are an endoscope capable of preventing a cleaning liquid from remaining on a surface of an observation optical system with a simpler configuration, and a method for manufacturing the endoscope. In the endoscope that includes a convex observation optical system provided at a distal end of an insertion portion and in which a cleaning liquid is ejected from an air and water supply nozzle, a distal end surface surrounding the observation optical system and having an uneven shape is provided.

### TECHNICAL FIELD

The present invention relates to an endoscope having a convex observation optical system and a method for manufacturing the endoscope.

The present application claims priority based on Japanese Patent Application No. 2020-051567 filed on Mar. 23, 2020, the entire contents of which are incorporated herein by reference.

### BACKGROUND ART

In the related art, in an endoscope, an observation optical system for imaging a subject is provided at a distal end of an insertion portion inserted into a body. A liquid used for cleaning tends to remain on a surface of the observation optical system. As described above, when the cleaning liquid remains on the observation optical system, it is difficult to obtain a clear image of the subject.

On the other hand, Patent Literature 1 discloses an endoscope capable of suppressing a protrusion amount of an observation window from the distal end of the insertion portion and improving a cleaning property and drainage property of the observation window.

Patent Literature 2 discloses an endoscope having high removing performance for the remaining liquid remaining on the observation window since a window surface of the observation window protrude from a flat portion of a distal end cover with a predetermined height, an inclined portion is provided between a periphery edge of the window surface of the observation window and the flat portion of the distal end cover, and at least a part of the flat portion of the distal end cover, the window surface of the observation window, or the inclined portion is made to have a surface property having high affinity with the cleaning liquid.

### CITATION LIST

### Patent Literature

Patent Literature 1: JP 2012-120701 A

Patent Literature 2: JP 2016-22006 A

### SUMMARY OF INVENTION

### Technical Problem

On the other hand, in order to improve a discovery rate of a lesion, a viewing angle of an observation optical system is required to be widened. With such a wide viewing angle, an objective lens of the observation optical system has a convex shape and a large diameter. Furthermore, in the observation optical system having such a convex shape, as described above, it is necessary to prevent the cleaning liquid from remaining on the surface of the observation optical system.

However, in the endoscope of Patent Literature 1, the protrusion amount of the observation window is suppressed, and the wide viewing angle of the observation optical system cannot be sufficiently achieved. Furthermore, in the endoscope of Patent Literature 2, the inclined portion is provided between the periphery edge of the window surface of the observation window and the flat portion of the distal end cover, and the surface property of the inclined portion is limited. Therefore, a configuration is complicated.

The present invention has been made in view of such circumstances, and an object of the present invention is to provide an endoscope including a convex observation optical system, the endoscope capable of preventing a cleaning liquid from remaining on a surface of the observation optical system with a simpler configuration, and a method for manufacturing the endoscope.

### Solution to Problem

According to the present invention, there is provided an endoscope that includes a convex observation optical system provided at a distal end of an insertion portion and in which a cleaning liquid is ejected from a nozzle, the endoscope including a distal end surface surrounding the observation optical system and having an uneven shape.

In the present invention, since the distal end surface surrounding the observation optical system has an uneven shape, wettability of the distal end surface is increased, and remaining liquid after cleaning easily spreads and moves to the distal end surface without gathering and staying at a boundary portion between the observation optical system and the distal end surface.

According to the present invention, there is provided a method for manufacturing an endoscope that includes a convex observation optical system provided at a distal end of an insertion portion and in which a cleaning liquid is ejected from a nozzle, the method including performing unevenness processing on a distal end surface surrounding the observation optical system.

In the present invention, for example, the distal end surface is made to have an uneven shape by unevenness processing such as blast processing or etching. Therefore, wettability of the distal end surface is increased, and remaining liquid after cleaning easily spreads and moves to the distal end surface without gathering and staying at a boundary portion between the observation optical system and the distal end surface.

According to the present invention, there is provided a method for manufacturing an endoscope that includes a convex observation optical system provided at a distal end of an insertion portion and in which a cleaning liquid is ejected from a nozzle, the method including forming a distal end surface surrounding the observation optical system and having an uneven shape by using a mold.

In the present invention, since the distal end surface made by using the mold has an uneven shape, wettability of the distal end surface is increased, and remaining liquid after cleaning easily spreads and moves to the distal end surface without gathering and staying at a boundary portion between the observation optical system and the distal end surface.

### Advantageous Effects of Invention

According to the present invention, in an endoscope including the convex observation optical system, it is possible to prevent the cleaning liquid from remaining on the surface of the observation optical system with a simpler configuration.

### BRIEF DESCRIPTION OF DRAWINGS

FIG. 1 is an external view of an endoscope according to a first embodiment of the present invention.

FIG. 2 is an external view of a distal end portion of the endoscope according to the first embodiment of the present invention.

FIG. 3 is a diagram illustrating an air and water supply nozzle of the endoscope according to the first embodiment of the present invention.

FIG. 4 is a result obtained by simulating a flow path of water ejected by an air and water supply nozzle in the endoscope according to the first embodiment of the present invention.

FIG. 5 is a result of simulating the flow path of water ejected by the air and water supply nozzle in the endoscope according to the first embodiment of the present invention.

FIG. 6 is a comparative diagram comparing a contact angle in a case where a water droplet adheres to a flat surface and a contact angle in a case where a water droplet adheres to a curved surface.

FIG. 7 is an explanatory view illustrating a flow of remaining water on an observation optical system and a distal end surface after water ejection from the air and water supply nozzle is completed in the endoscope according to the first embodiment of the present invention.

FIG. 8 is a view illustrating a modified example of a distal end surface in the endoscope according to the first embodiment of the present invention.

FIG. 9 is a cross-sectional view taken along line IX-IX of FIG. 8.

FIG. 10 is a view illustrating a distal end surface of an endoscope according to a second embodiment of the present invention.

FIG. 11 is an enlarged cross-sectional view taken along line XI-XI of FIG. 10.

FIG. 12 is a view illustrating a distal end surface of an endoscope according to a third embodiment of the present invention.

FIG. 13 is an enlarged cross-sectional view taken along line XIII-XIII of FIG. 12.

FIG. 14 is a view illustrating a distal end surface of an endoscope according to a fourth embodiment of the present invention.

FIG. 15 is an enlarged cross-sectional view taken along line XV-XV of FIG. 14.

FIG. 16 is an external view illustrating a distal end portion of an endoscope according to a fifth embodiment of the present invention.

FIG. 17 is a cross-sectional view taken along line XVII-XVII of FIG. 16.

### DESCRIPTION OF EMBODIMENTS

Hereinafter, an endoscope according to embodiments of the present invention will be described in detail with reference to the drawings.

### First Embodiment

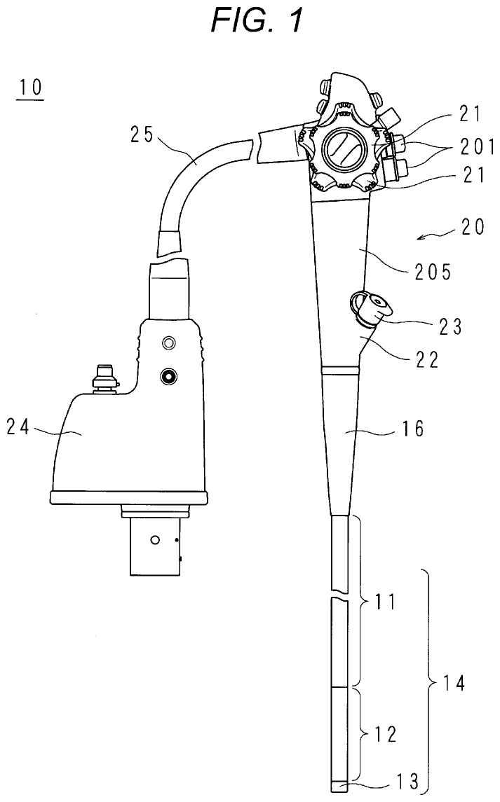

FIG. 1 is an external view of an endoscope 10 according to the first embodiment of the present invention. The endoscope 10 according to the embodiment includes an insertion portion 14, an operation unit 20, a universal cord 25, and a connector unit 24. The operation unit 20 includes a button 201 and an angulation knob 21, which are operated by a user, and a channel inlet 22 provided in a case 205 having a substantially cylindrical shape. A biopsy valve 23 having an insertion port for inserting a treatment tool or the like is attached to the channel inlet 22.

The insertion portion 14 is inserted into a body of a subject. The insertion portion 14 is long and includes a distal end portion 13, a bending portion 12, and a soft portion 11 in this order from one end of the distal end. The other end of the insertion portion 14 is connected to the operation unit 20 via a bend preventing portion 16. The bending portion 12 is bent according to an operation of the angulation knob 21.

In the following description, a longitudinal direction of the insertion portion 14 is also referred to as an insertion direction. Furthermore, in the insertion portion 14, the one end side close to the operation unit 20 is referred to as an operation unit side, and the other end side close to the distal end portion 13 is also referred to as a distal end portion side.

The universal cord 25 is long, and has one end connected to the operation unit 20 and the other end connected to the connector unit 24. The universal cord 25 is soft. The connector unit 24 is connected to a processor for an endoscope (not illustrated), a light source device, a display device, an air and water supply device, and the like. By appropriately operating the operation unit 20, a cleaning fluid (air or water) sent through the connector unit 24 is sent to the distal end portion 13 via the bend preventing portion 16.

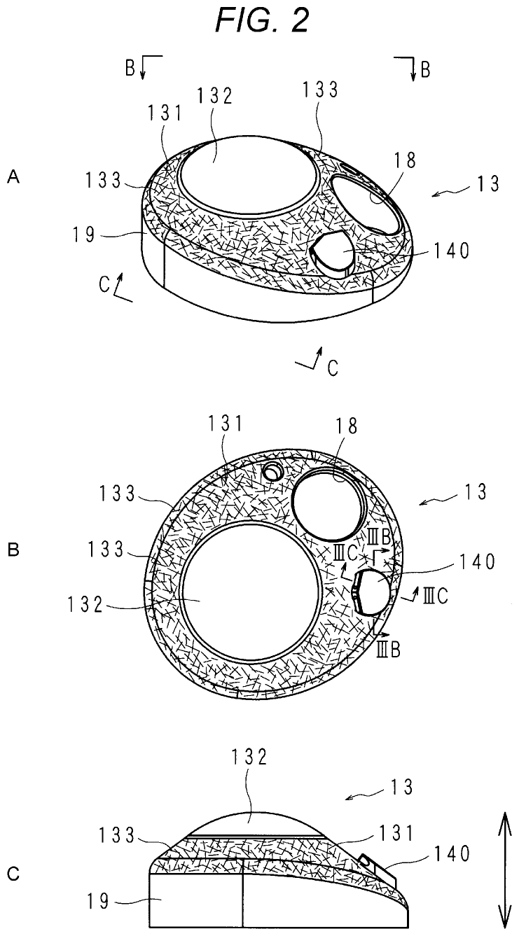

FIG. 2 is an external view of the distal end portion 13 of the endoscope 10 according to the first embodiment of the present invention. FIG. 2A is a perspective view of the distal end portion 13, FIG. 2B is a view taken along line B-B of FIG. 2A, and FIG. 2C is a view taken along line C-C of FIG. 2A.

The distal end portion 13 is substantially elliptical in cross section, and has the distal end protruding in a substantially conical shape. A distal end surface 131 of the distal end portion 13 is provided with an observation optical system 132, an air and water supply nozzle 140, a channel outlet 18 (suction hole), and the like.

Furthermore, the distal end portion 13 has a cylindrical accommodation cylinder 19 which accommodates an image sensor (not illustrated) or the like that captures image light of the subject via the observation optical system 132 to perform imaging, and the distal end surface 131 of the distal end portion 13 extends from an edge of the accommodation cylinder 19. Sending paths of air and water ejected through the air and water supply nozzle 140 are formed in the accommodation cylinder 19, the bending portion 12, and the soft portion 11.

The observation optical system 132 is provided at the central part of the distal end surface 131 of the distal end portion 13, and an objective lens is a circular convex lens. Furthermore, in the distal end surface 131 of the distal end portion 13, the air and water supply nozzle 140 and the channel outlet 18 are provided around the observation optical system 132.

The distal end surface 131 of the distal end portion 13 surrounds the observation optical system 132 and has an appearance of a substantially truncated cone. That is, the distal end surface 131 is an inclined surface extending in a tangential direction from the edge portion of the observation optical system 132 and inclined with respect to the insertion direction. The air and water supply nozzle 140 is provided on the distal end surface 131, and the channel outlet 18 is opened.

The distal end surface 131 has an uneven shape. More specifically, a plurality of recesses 133 are randomly formed on the distal end surface 131. A distance between the recesses 133 is, for example, from 0.1 mm to 0.35 mm, and a depth of the recesses 133 is, for example, 0.005 mm to 0.02 mm.

In a manufacturing process of the endoscope 10, for example, the distal end surface 131 is subjected to unevenness processing. As a result, each of the recesses 133 is formed on the distal end surface 131, and the distal end surface 131 becomes uneven as a whole. Examples of the unevenness processing include blast processing, etching, and hairline finishing.

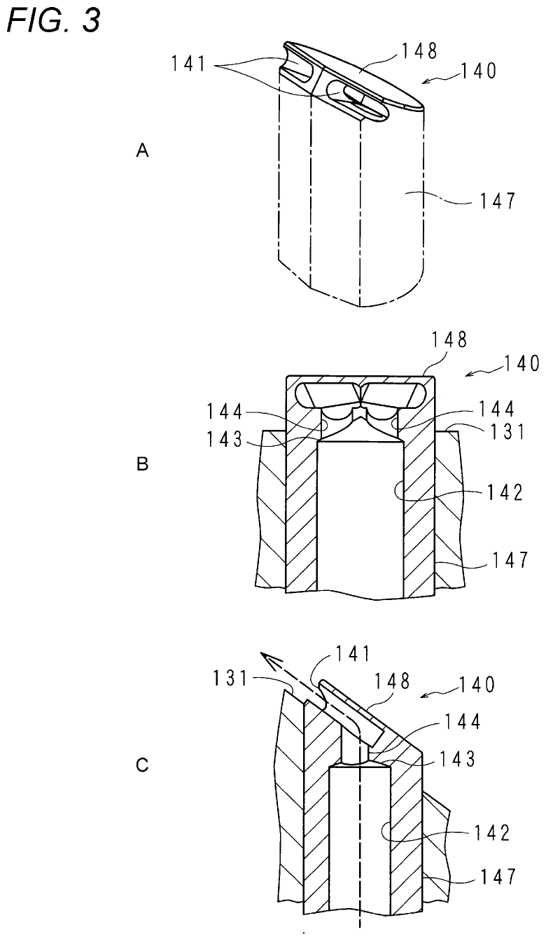

FIG. 3 is a diagram illustrating the air and water supply nozzle 140 of the endoscope 10 according to the first embodiment of the present invention. FIG. 3A is a perspective view illustrating an appearance of the air and water supply nozzle 140, FIG. 3B is a cross-sectional view taken along line IIIB-IIIB of FIG. 2B, and FIG. 3C is a cross-sectional view taken along line IIIC-IIIC of FIG. 2B.

The air and water supply nozzle 140 ejects air or liquid toward the observation optical system 132 along the distal end surface 131. Hereinafter, a case where the air and water supply nozzle 140 ejects water will be described.

The air and water supply nozzle 140 has a plurality of outlets 141 from which water is ejected. The water is ejected toward the observation optical system 132 through each of the outlets 141.

In the present embodiment, a case where the air and water supply nozzle 140 has two outlets 141 will be described as an example. However, the present invention is not limited to this, and may be configured to have three or more outlets 141.

Each of the outlets 141 is open in different directions. That is, the water is ejected through each of the outlets 141 in a direction that does not intersect with each other. Each of the outlets 141 has an oval shape with a direction along the distal end surface 131 as a long axis direction. Most part of the air and water supply nozzle 140 (dashed line portion in FIG. 3A) is inserted into a hole formed on the distal end surface 131 and fixed.

As described above, the observation optical system 132 is provided at the distal end of the distal end portion 13, the distal end surface 131 forms a slope so as to surround the circular edge of the observation optical system 132, and the air and water supply nozzle 140 is provided on the distal end surface 131 separated from the observation optical system 132. That is, in the endoscope 10 according to the first embodiment of the present invention, in a longitudinal direction of the insertion portion 14 (refer to an arrow in FIG. 2C), the air and water supply nozzle 140 is disposed at a position closer to the other end of the insertion portion 14 (operation unit 20 side) rather than the observation optical system 132.

Since the objective lens of the observation optical system 132 is a convex lens and has a wide viewing angle (180 degrees or more), in a case where the air and water supply nozzle 140 is disposed at the same position as that of the observation optical system 132 in the longitudinal direction of the insertion portion 14, the air and water supply nozzle 140 appears in the captured image of the observation optical system 132. However, in the endoscope 10 according to the first embodiment of the present invention, as described above, the air and water supply nozzle 140 is disposed at a position closer to the other end of the insertion portion 14 than the observation optical system 132. Therefore, the air and water supply nozzle 140 does not appear in the captured image of the observation optical system 132, and does not interfere with the captured image of the observation optical system 132.

The air and water supply nozzle 140 has a cylindrical portion 147 and a lid portion 148 that seals one open end of the cylindrical portion 147. The lid portion 148 and the cylindrical portion 147 are integrally formed. The lid portion 148 has a substantially disk shape and is inclined with respect to the longitudinal direction (axial direction) of the cylindrical portion 147.

In the air and water supply nozzle 140, the outlet 141 is formed at one end portion on the lid portion 148 side. The air and water supply nozzle 140 has a connecting pipe portion 142 extending along the longitudinal direction of the cylindrical portion 147 inside the cylindrical portion 147. The connecting pipe portion 142 sends the water sent through the connector unit 24 and the bend preventing portion 16 to each of the outlets 141. That is, the water flowing into the connecting pipe portion 142 through the opening at one end of the connecting pipe portion 142 is sent to the outlet 141 on the other end side (lid portion 148 side).

In the other end portion (end portion on the lid portion 148 side) on the downstream side of the connecting pipe portion 142, a divergence portion 144 that divides the flow of the water flowing through the connecting pipe portion 142 into the number of the outlets 141 is provided. That is, the downstream side of the connecting pipe portion 142 is divided into two flow paths (flow divergence portions 144) having a diameter smaller than that of the connecting pipe portion 142. Each of the divergence portions 144 is provided so as to correspond to any one of the outlets 141, and the water flowing into each of the divergence portions 144 flows to the corresponding outlet 141 to be ejected.

Furthermore, a funnel-shaped or tapered diameter-reduced portion 143 is formed on the downstream side of the connecting pipe portion 142 and on the upstream side of the divergence portion 144. That is, the diameter-reduced portion 143 is formed between the divergence portion 144 and the other end portion of the connecting pipe portion 142, and the diameter of the connecting pipe portion 142 is reduced at the diameter-reduced portion 143.

Accordingly, a pressure of the water flowing into each of the divergence portions 144 through the diameter-reduced portion 143 is reduced, and a flow speed is increased. The water having a higher flow speed flows out into a space wider than the divergence portion 144 (refer to FIGS. 3B and 3C), and flows toward the outlet 141. At this time, the water forms a vortex having vectors in various directions, and then is ejected from the outlet 141. Therefore, the water ejected from each of the outlets 141 spreads over a wide range, and ejection force and a range at the time of the ejection can be secured. FIG. 3C illustrates the flow path of the water with a broken line.

As described above, in the air and water supply nozzle 140, the directions of the outlets 141 are different from each other, and the water is ejected from each of the outlets 141 in directions that do not intersect with each other. That is, in a case where the water is ejected linearly through the outlet 141 and maintains the linearity even after the ejection, each of the outlets 141 is provided such that the water from each of the outlets 141 does not intersect with each other.

With such a configuration, the endoscope 10 according to the present embodiment can clearly clean portions of the convex observation optical system 132 from a portion on the air and water supply nozzle 140 side with which the ejected water comes into direct contact to the side opposite to the portion on the air and water supply nozzle 140 by using one air and water supply nozzle 140 Hereinafter, in the observation optical system 132, the portion on the air and water supply nozzle 140 side with which the ejected water comes into direct contact is referred to as a nozzle side portion, and a side opposite to the nozzle side portion is referred to as a nozzle-opposite portion.

In general, the fluid flowing near the wall surface is attracted to the wall surface by an effect of fluid viscosity (refer to as Coanda effect). Due to the Coanda effect, in a case where the fluid flows along the surface (curved surface) of the convex lens, the fluid exhibits a behavior of concentrating toward the center of the curved surface. The fluid concentrated in this way is separated from the curved surface of the convex lens due to weight and inertia of the fluid. Therefore, in a case where the water is ejected to the nozzle side portion of the observation optical system from one air and water supply nozzle (outlet), the water does not reach the nozzle-opposite portion of the observation optical system, and the observation optical system is insufficiently cleaned.

For example, even when the outlet of the air and water supply nozzle is widened and the water is ejected over a wide range of the observation optical system, the water ejected from the outlet is concentrated toward the center of the observation optical system without changes, and as described above, the water is separated from the curved surface of the observation optical system.

Furthermore, even in a case where the air and water supply nozzle has a plurality of outlets and water is ejected from a plurality of the outlets, the water from one outlet starts to spread after being ejected, and is merged with the water from the other outlets. Therefore, as described above, the water is concentrated toward the center of the observation optical system, and is separated from the curved surface of the observation optical system.

On the other hand, in the endoscope 10 according to the first embodiment of the present invention, two outlets 141 are provided to have the directions of outlets 141 different from each other so that the ejected water does not intersect with each other.

Therefore, it is possible to prevent the water ejected from one outlet 141 from being merged with the water ejected from the other outlet 141. Accordingly, since it is possible to prevent the water from being concentrated toward the center of the observation optical system 132 and being separated from the curved surface of the observation optical system 132 in advance, the water can flow up to and clean the nozzle-opposite portion of the observation optical system 132.

Furthermore, since the water from each of the outlets 141 approaches the center of the observation optical system 132 due to the Coanda effect, the entire observation optical system 132 can be sufficiently cleaned by including the center portion of the observation optical system 132.

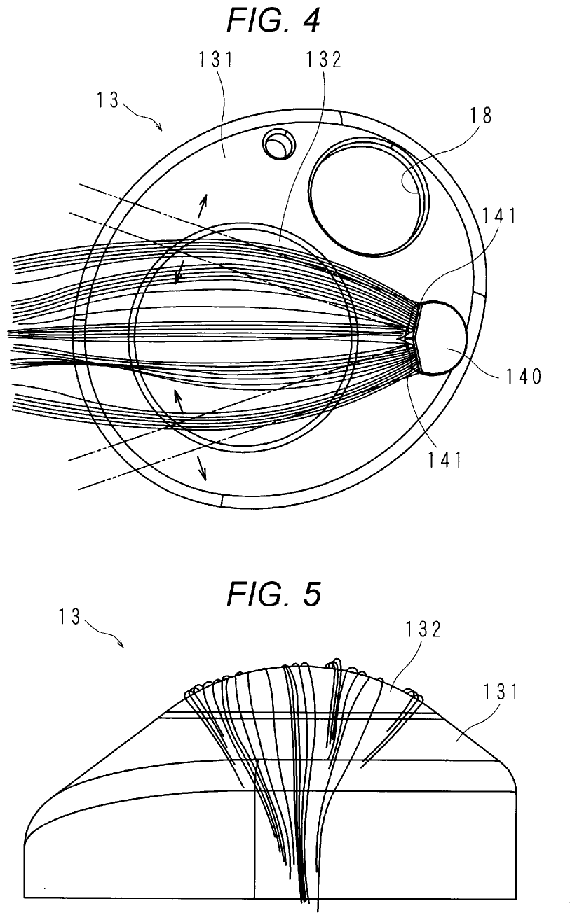

FIGS. 4 and 5 are the results obtained by simulating the flow path of the water ejected from the air and water supply nozzle 140 in the endoscope 10 according to the first embodiment of the present invention. FIG. 4 mainly illustrates the upstream side of the flow path, and FIG. 5 mainly illustrates the downstream side. That is, FIG. 5 illustrates the flow path on the nozzle-opposite portion of the observation optical system 132. Furthermore, in FIG. 4, a two-dot chain line indicates the direction of each of the outlets 141, and a solid line indicates the flow path of the water ejected from the outlet 141. Note that for convenience, the uneven shape of the distal end surface 131 is not illustrated in FIGS. 4 and 5.

As can be seen from FIGS. 4 and 5, in the endoscope 10 according to the first embodiment of the present invention, the water ejected from one outlet 141 starts to spread after being ejected (refer to an arrow in FIG. 4), but the water ejected from one outlet 141 is hardly merged with the water ejected from the other outlet 141, it is not found that the water is concentrated at the center of the observation optical system 132, and the water is not separated from the curved surface of the observation optical system 132. Furthermore, the water ejected from the air and water supply nozzle 140 flows up to the nozzle-opposite portion of the observation optical system 132 (refer to FIG. 5). Therefore, the entire observation optical system 132 can be sufficiently cleaned.

Furthermore, since the observation optical system 132 is made of glass and the distal end surface 131 is made of a resin, and the contact angle between the glass and the liquid (water) is generally about half the contact angle between the resin and the liquid, wettability (hydrophilicity) of the observation optical system 132 is better than the wettability of the distal end surface 131. That is, the water more easily spreads and moves on the observation optical system 132 than the distal end surface 131. Moreover, in the observation optical system 132, since the objective lens is a convex lens and has a curved surface, the wettability with a water droplet increases.

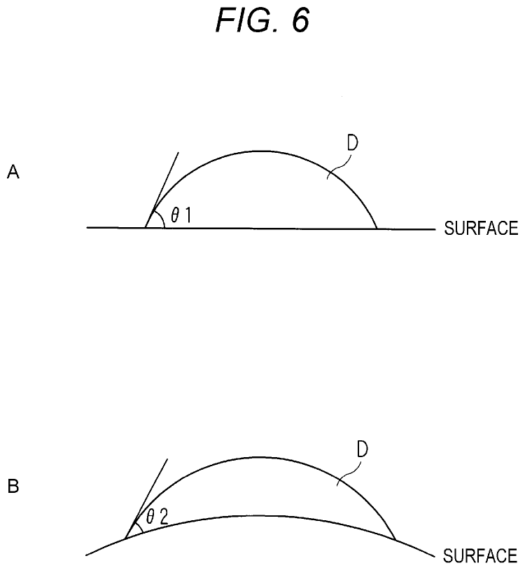

FIG. 6 is a comparative diagram comparing a contact angle in a case where a water droplet adheres to a flat surface and a contact angle in a case where a water droplet adheres to a curved surface. FIG. 6A illustrates a case where the water droplet adheres to the flat surface, and FIG. 6B illustrates a case where the water droplet adheres to the curved surface.

As can be seen from FIG. 6, a contact angle θ2 in a case where the water droplet adheres to the curved surface is smaller than a contact angle θ1 in a case where the water droplet adheres to the flat surface, and the wettability increases. Therefore, the water droplet more easily spreads and moves on the observation optical system 132.

However, as described above, since the contact angle between the water and the resin is as large as twice the contact angle between the water and the glass, and the wettability is poor, mobility of the water on the resin is poor. Accordingly, after the water ejection from the air and water supply nozzle 140 is completed, there is a possibility that the remaining water (remaining liquid) remaining on the observation optical system 132 moves on the surface of the observation optical system 132, gathers at a boundary portion between the observation optical system 132 and the distal end surface 131, and stays at the boundary portion. In such a case, imaging of the subject is hindered, and it is difficult to capture a clear image.

On the other hand, in the endoscope 10 according to the first embodiment, as described above, the distal end surface 131 has the uneven shape, and thus, it is possible to prevent the water droplet from remaining at the boundary portion between the observation optical system 132 and the distal end surface 131. The details will be described below.

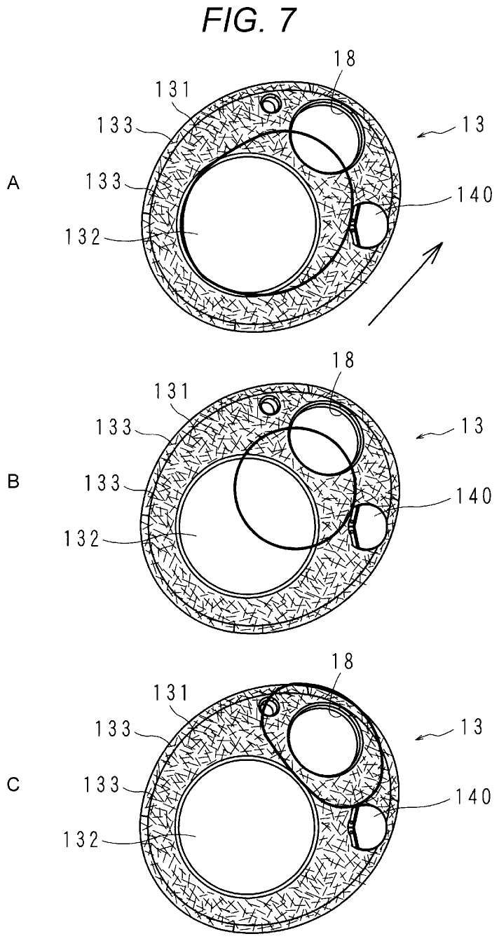

FIG. 7 is an explanatory view illustrating a flow of the remaining water on the observation optical system 132 and the distal end surface 131 after the water ejection from an air and water supply nozzle 140 is completed in the endoscope 10 according to the first embodiment of the present invention. FIGS. 7A, 7B and 7C illustrate the flow of the remaining water over time. In FIGS. 7A, 7B, and 7C, thick solid circles indicate the remaining water.

As described above, the wettability between the water droplet and the resin is worse than the wettability between the water droplet and the glass, and there is a possibility that the water droplet hardly spreads and hardly moves on the distal end surface 131 made of the resin.

However, in the endoscope 10 according to the first embodiment of the present invention, since the distal end surface 131 has an uneven shape, a contact area between the distal end surface 131 and the water droplet increases, and thus the hydrophilicity is increased. Therefore, the liquid droplet easily spreads and moves on the distal end surface 131.

Specifically, after the water ejection from the air and water supply nozzle 140 is completed, as illustrated in FIG. 7A, the remaining water remaining at the center portion of the distal end surface 131 including the observation optical system 132 starts to flow in a gravity direction (arrow direction in FIG. 7A). At this time, the remaining water forms one aggregate as a whole due to surface tension.

The remaining water moves, on the surface of the observation optical system 132, to the boundary portion between the observation optical system 132 and the distal end surface 131, that is, the edge of the distal end surface 131 while maintaining the state of the aggregate due to the surface tension. The hydrophilicity of the distal end surface 131 is increased by the uneven shape, and the remaining water reaching the edge of the distal end surface 131 spreads and moves on the distal end surface 131 as it is without staying (refer to FIGS. 7B and 7C). The remaining water moves up to the edge of the distal end surface 131 and flows down in this way.

That is, after the water ejection from the air and water supply nozzle 140 is completed, the remaining water remaining at the center portion of the distal end surface 131 including the observation optical system 132 starts to move while maintaining the state of one aggregate, and moves from the observation optical system 132 to the distal end surface 131 without staying at the boundary portion between the observation optical system 132 and the distal end surface 131. Therefore, it is difficult for the water droplet to remain on the observation optical system 132.

As described above, the endoscope 10 according to the first embodiment can prevent the cleaning water from remaining on the surface of the observation optical system 132 after ejecting the cleaning water with a simple configuration in which the distal end surface 131 has the uneven shape.

Note that as described above, a case where only the distal end surface 131 has the uneven shape has been described, but the present invention is not limited to this. For example, in addition to the distal end surface 131, the accommodation cylinder 19 (surface) may also be configured to have an uneven shape.

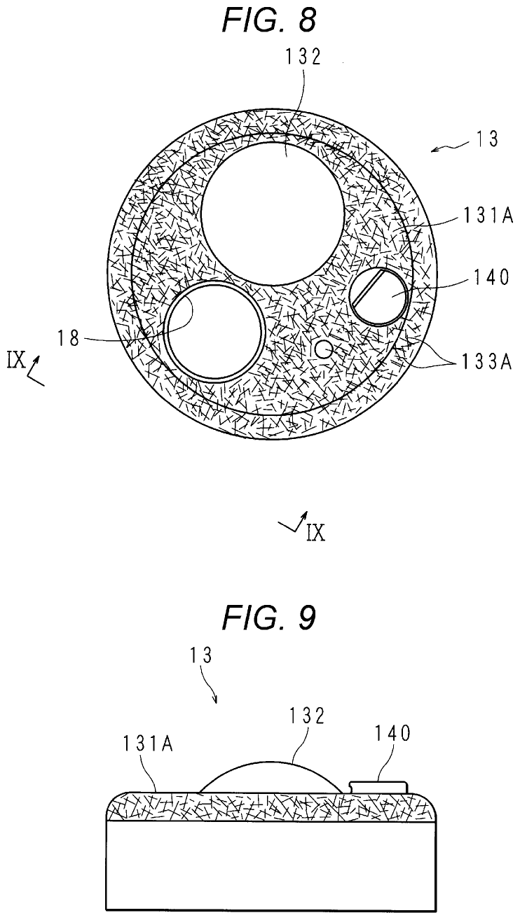

In the above description, a case where the distal end surface 131 has a substantially truncated cone shape, and is inclined with respect to the longitudinal direction of the insertion portion 14 has been described as an example, but the present invention is not limited to this. FIG. 8 is a view illustrating a modified example of the distal end surface 131 in the endoscope 10 according to the first embodiment of the present invention, and FIG. 9 is a view taken along line IX-IX of FIG. 8. Hereinafter, the modified example of the distal end surface 131 is referred to as a distal end surface 131A.

The distal end surface 131A is a flat surface orthogonal to the longitudinal direction of the insertion portion 14 and has an uneven shape. Furthermore, the distal end surface 131A is provided with the observation optical system 132, the air and water supply nozzle 140, and the channel outlet 18. As illustrated in FIGS. 8 and 9, even in a case where the distal end surface 131A is a flat surface, it goes without saying that the effect described above is obtained.

### Second Embodiment

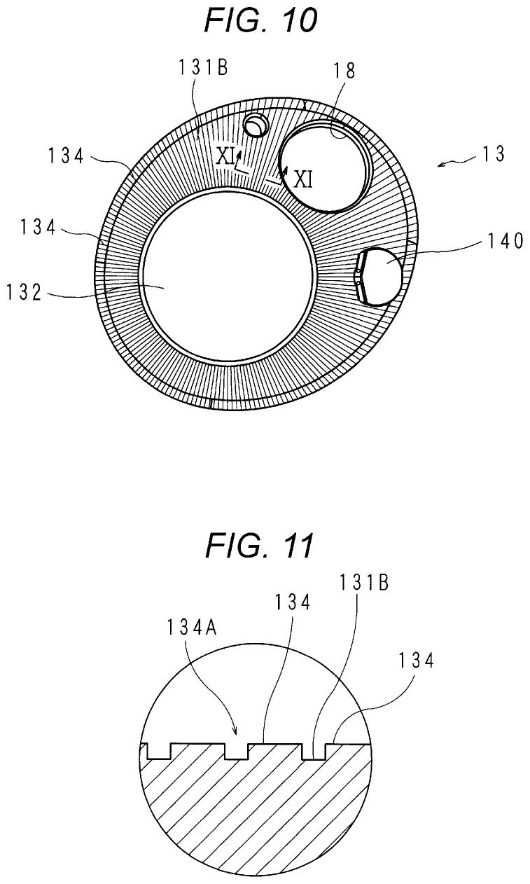

FIG. 10 is a view illustrating a distal end surface 131B in an endoscope 10 according to the second embodiment of the present invention, and FIG. 11 is an enlarged cross-sectional view taken along line XI-XI of FIG. 10.

An observation optical system 132 is provided at the center portion of the distal end surface 131B as in the first embodiment. That is, the distal end surface 131B surrounds the observation optical system 132. The distal end surface 131B is an inclined surface extending in a tangential direction from the edge portion of the observation optical system 132 and inclined with respect to an insertion direction, the distal end surface 131B having a substantially truncated cone shape. An air and water supply nozzle 140 is provided on the distal end surface 131B, and a channel outlet 18 is opened.

The distal end surface 131B has an uneven shape. More specifically, a plurality of protrusions 134 are formed at equal intervals on the distal end surface 131B. Each of the protrusions 134 extends linearly in a direction away from a proximal side of the observation optical system 132. That is, a plurality of the protrusions 134 are radially formed around the observation optical system 132.

In a manufacturing process of the endoscope 10, for example, the distal end surface 131B is formed by a mold. A distance between the protrusions 134 is, for example, from 0.3 mm to 0.5 mm, a height of the protrusions 134 is, for example, 0.1 mm, and a width of the protrusions 134 is, for example, 0.3 mm.

In this manner, a plurality of the protrusions 134 are formed on the distal end surface 131B, and the distal end surface 131B becomes uneven as a whole. Furthermore, since a recess is relatively formed between the protrusions 134, a groove 134A is formed (refer to FIG. 11).

As described above, a case where a plurality of the protrusions 134 are formed on the distal end surface 131B, and the protrusions 134 configure the groove 134A, but the present invention is not limited to this. The recess having the same shape as that of the protrusion 134 may be formed on the distal end surface 131B.

In the endoscope 10 according to the second embodiment, since the distal end surface 131B has an uneven shape, a contact area between the distal end surface 131B and the remaining water increases, and thus hydrophilicity is increased. Therefore, remaining water easily spreads and moves on the distal end surface 131B.

Accordingly, after the water ejection from the air and water supply nozzle 140 is completed, the remaining water remaining at the center portion of the distal end surface 131B including the observation optical system 132 moves while maintaining the state of one aggregate, and moves to the distal end surface 131B without staying at a boundary portion between the observation optical system 132 and the distal end surface 131B. Therefore, it is difficult for a water droplet to remain on the observation optical system 132.

Moreover, in the endoscope 10 according to the second embodiment, the adjacent protrusions 134 extend in the same direction to form the groove 134A. This causes the remaining water to move. Therefore, it is possible to prevent the movement of the remaining water on the distal end surface 131B from being unnecessarily delayed.

Furthermore, a user of the endoscope 10 can suck the remaining water on the distal end surface 131B via the channel outlet 18 by appropriately operating the button 201 (refer to FIG. 1). On the other hand, in the endoscope 10 according to the second embodiment, as described above, a plurality of the protrusions 134 or the grooves 134A radially extend around the observation optical system 132, and a part of the protrusions 134 or the grooves 134A extends from the observation optical system 132 to the channel outlet 18.

Therefore, the protrusions 134 or the grooves 134A can guide the remaining water on the distal end surface 131B (observation optical system 132) to the channel outlet 18, and the remaining water from the channel outlet 18 is more efficiently sucked.

The protrusion 134 protruding from the distal end surface 131B may have a constant dimension (width) in a direction intersecting with a protruding direction, or may be configured so that the width becomes narrower as it is closer to the distal end. In a case where the width is narrowed as it is closer to the distal end, it is easy to perform removal from the mold at the time of manufacturing using a mold.

The same portions as those in the first embodiment are denoted by the same reference numerals, and detailed description thereof will be omitted.

### Third Embodiment

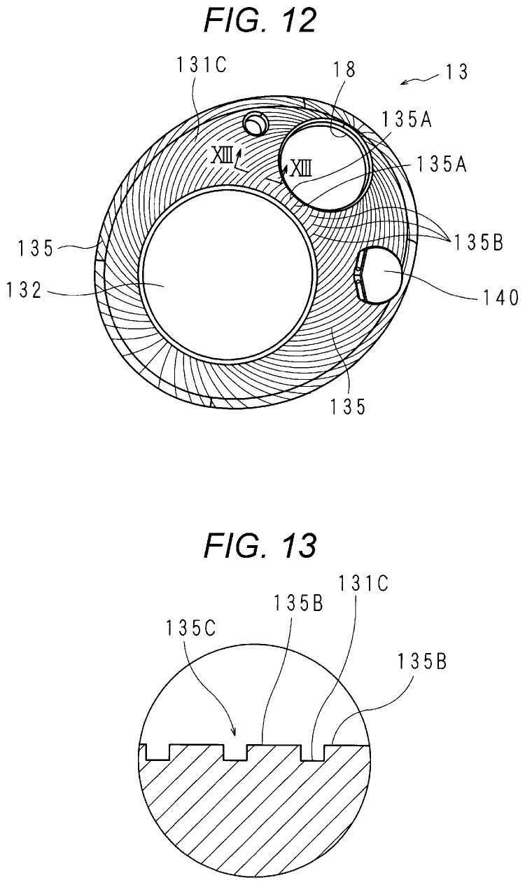

FIG. 12 is a view illustrating a distal end surface 131C in an endoscope 10 according to the third embodiment of the present invention, and FIG. 13 is an enlarged cross-sectional view taken along line XIII-XIII of FIG. 12.

The distal end surface 131C surrounds an observation optical system 132 provided at the center portion, and is an inclined surface extending in a tangential direction from the edge portion of the observation optical system 132 and inclined with respect to an insertion direction, the distal end surface 131C having a substantially truncated cone shape. An air and water supply nozzle 140 is provided on the distal end surface 131C, and a channel outlet 18 is opened.

The distal end surface 131C has an uneven shape. More specifically, a plurality of protrusions 135 are formed at substantially equal intervals on the distal end surface 131C. Each of the protrusions 135 extends linearly or in a curved manner in a direction away from a proximal side of the observation optical system 132. A plurality of the protrusions 135 include a linear protrusion 135A or a curved protrusion 135B extending from the observation optical system 132 to the channel outlet 18. The protrusion 135B is disposed in parallel in a direction orthogonal to the protrusion 135A, and a length and a curvature increase as a distance from the protrusion 135A increases.

In a manufacturing process of the endoscope 10, for example, the distal end surface 131C is formed by a mold. As a result, a plurality of the protrusions 135 are formed on the distal end surface 131C, and the distal end surface 131C becomes uneven as a whole. Furthermore, since a recess is relatively formed between the protrusions 135, a groove 135C is formed. In other words, the groove 135C extending from the observation optical system 132 to the channel outlet 18 is formed on the distal end surface 131C (refer to FIG. 12).

As described above, a case where a plurality of the protrusions 135 are formed on the distal end surface 131C, and the protrusions 135 configure the groove 135C, but the present invention is not limited to this. The recess having the same shape as that of the protrusion 135 may be formed on the distal end surface 131C.

In the endoscope 10 according to the third embodiment, since the distal end surface 131C has an uneven shape, a contact area between the distal end surface 131C and the remaining water increases, and thus hydrophilicity is increased. Therefore, the remaining water easily spreads and moves on the distal end surface 131C.

Accordingly, after the water ejection from the air and water supply nozzle 140 is completed, the remaining water remaining at the center portion of the distal end surface 131C including the observation optical system 132 moves while maintaining the state of one aggregate, and moves to the distal end surface 131C without staying at a boundary portion between the observation optical system 132 and the distal end surface 131C. Therefore, it is difficult for a water droplet to remain on the observation optical system 132.

Moreover, in the endoscope 10 according to the third embodiment, the adjacent protrusions 135 form the groove 135C and extend, thereby guiding the movement of the remaining water. Therefore, it is possible to prevent the movement of the remaining water on the distal end surface 131C from being unnecessarily delayed.

Furthermore, a user of the endoscope 10 can suck the remaining water on the distal end surface 131C via the channel outlet 18 by appropriately operating the button 201 (refer to FIG. 1). On the other hand, in the endoscope 10 according to the third embodiment, as described above, the linear protrusions 135A or the curved protrusion 135B (groove 135C) extends from the observation optical system 132 to the channel outlet 18.

Therefore, the protrusion 135A and the protrusion 135B (groove 135C) can guide the remaining water on the distal end surface 131C (observation optical system 132) to the channel outlet 18, and the remaining water from the channel outlet 18 is more efficiently sucked.

The same portions as those in the first embodiment are denoted by the same reference numerals, and detailed description thereof will be omitted.

### Fourth Embodiment

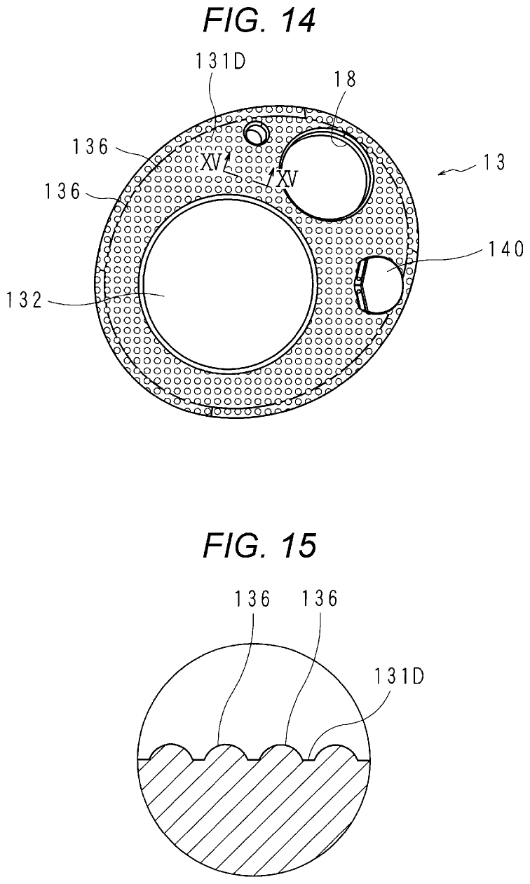

FIG. 14 is a view illustrating a distal end surface 131D in an endoscope 10 according to the fourth embodiment of the present invention, and FIG. 15 is an enlarged cross-sectional view taken along line XV-XV of FIG. 14.

The distal end surface 131D is provided with an observation optical system 132 at the center portion thereof, and is an inclined surface extending in a tangential direction from the edge portion of the observation optical system 132 and inclined with respect to an insertion direction, the distal end surface 131D having a substantially truncated cone shape. An air and water supply nozzle 140 is provided on the distal end surface 131D, and a channel outlet 18 is opened.

The distal end surface 131D has an uneven shape. More specifically, a plurality of protrusions 136 are formed on the distal end surface 131D. Each of the protrusions 136 has a dot shape.

In a manufacturing process of the endoscope 10, for example, the distal end surface 131D is formed by a mold. As a result, a plurality of the protrusions 136 are formed on the distal end surface 131D, and the distal end surface 131D becomes uneven as a whole.

Furthermore, the present invention is not limited to this. A recess having the same shape as that of the protrusion 136 may be formed on the distal end surface 131D.

In the endoscope 10 according to the fourth embodiment, since the distal end surface 131D has an uneven shape, a contact area between the distal end surface 131D and the remaining water increases. Therefore, hydrophilicity is increased, and the remaining water easily spreads and moves on the distal end surface 131D.

Accordingly, after the water ejection from the air and water supply nozzle 140 is completed, the remaining water remaining at the center portion of the distal end surface 131D including the observation optical system 132 moves to the distal end surface 131D without staying at a boundary portion between the observation optical system 132 and the distal end surface 131D while maintaining the state of one aggregate. Therefore, it is difficult for a water droplet to remain on the observation optical system 132.

In the above description, a case where the observation optical system 132 is made of glass and the distal end surfaces 131,131A, 131B, 131C, and 131D (hereinafter, simply referred to as distal end surface 131) are made of a resin has been described as an example, but the present invention is not limited to this. For example, the observation optical system 132 may be made of a resin. In this case, it goes without saying that the effect described above is obtained.

That is, in a case where the observation optical system 132 and the distal end surface 131 are made of a resin, since the distal end surface 131 has an uneven shape, wettability of the distal end surface 131 is better than the wettability of the observation optical system 132. Therefore, the remaining water moves to the distal end surface 131C without staying at a boundary portion between the observation optical system 132 and the distal end surface 131C. Therefore, it is difficult for a water droplet to remain on the observation optical system 132.

### Fifth Embodiment

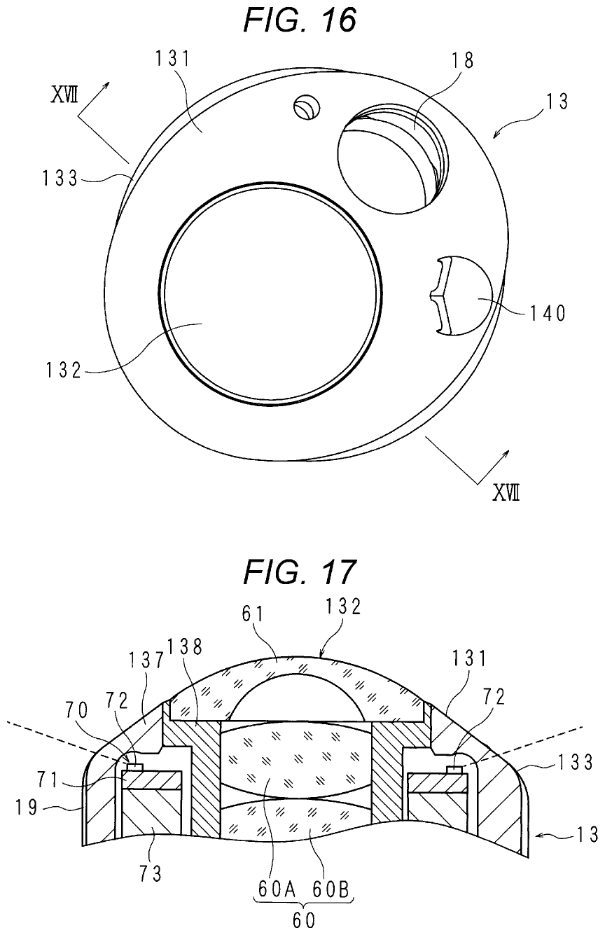

FIG. 16 is an external view illustrating a distal end portion 13 in an endoscope 10 according to the fifth embodiment of the present invention, and FIG. 17 is a cross-sectional view taken along line XVII-XVII of FIG. 16.

An annular light distribution lens 137 is fitted into the accommodation cylinder 19 of the distal end portion 13. In the light distribution lens 137, one end portion on the distal end side of the distal end portion 13 is bent inward and reduced in diameter to form a diameter-reduced portion. Accordingly, an outer surface of the one end portion of the light distribution lens 137 forms an inclined surface with respect to an axial center of the accommodation cylinder 19. That is, in the endoscope 10 according to the fifth embodiment of the present invention, the outer surface of one end portion of the light distribution lens 137 forms a distal end surface 131 of the distal end portion 13.

An observation optical system 132 is provided on a center side of the light distribution lens 137. The observation optical system 132 includes an observation window 61 and a plurality of lenses 60. The observation window 61 is a wide-angle objective lens having a substantially hemispherical shape. A plurality of the lenses 60 include a lens (not illustrated) together with a lens 60A and a lens 60B. By configuring the observation optical system 132 including the observation window 61 and a plurality of the lenses 60, imaging can be performed at a viewing angle of 180° or more.

Furthermore, a lens-holding barrel 138 that holds the observation window 61 and a plurality of the lenses 60 is provided on the center side of the light distribution lens 137. The lens-holding barrel 138 has a cylindrical shape extending along an axial center of the light distribution lens 137. One end side of the lens-holding barrel 138 is enlarged in diameter, and an end surface on one end side is exposed from the distal end surface 131 and surrounded by a side edge of one end portion of the light distribution lens 137.

The observation window 61 and a plurality of the lenses 60 are disposed on the axial center of the lens-holding barrel 138. The observation window 61 is fitted into an enlarged-diameter portion of the lens-holding barrel 138, and periphery edge portions of a plurality of the lenses 60 are interposed around an inner surface of the lens-holding barrel 138 on the inner side of the observation window 61 so that the lenses 60 are adjacent to each other. The observation window 61 is exposed outward from the distal end surface 131. The exposed portion of the observation window 61 is surrounded by the lens-holding barrel 138 and is continuous with one end of the lens-holding barrel 138.

Inside the accommodation cylinder 19, the illumination unit 70 is incorporated between the lens-holding barrel 138 and the light distribution lens 137. That is, the illumination unit 70 is circumferentially provided in the vicinity of an outer circumferential surface of the lens-holding barrel 138.

The illumination unit 70 includes a cylindrical illumination holder 73 surrounding the circumference of the lens-holding barrel 138, an annular substrate 71 provided on an end surface of the illumination holder 73, and a plurality of LEDs 72 mounted on one surface of the substrate 71 opposed to the light distribution lens 137.

The LEDs 72 are disposed at substantially equal intervals in a circumferential direction of the substrate 71. Light emitted from each of the LEDs 72 is emitted through the light distribution lens 137 and illuminates an imaging visual field of the observation optical system 132. The LED 72 is, for example, a white LED that emits white light. Furthermore, the LED 72 may be another light emitting element such as an LD.

Broken lines in FIG. 17 indicate a light distribution range of the LED 72. The light emitted by the LED 72 is incident on the diameter-reduced portion and the bending portion of one end portion of the light distribution lens 137 in a wide range and greatly spreads. Note that at one end portion of the light distribution lens 137, a recess is formed on an inner surface of the bending portion. The light distribution of the LED 72 is radiated in a wide range due to the action of the recess.

Moreover, also in the endoscope 10 according to the fifth embodiment, the distal end surface 131 has an uneven shape. Therefore, the light emitted from the LED 72 is incident on the light distribution lens 137, diffused at the distal end surface 131, and emitted.

Accordingly, in the endoscope 10 according to the fifth embodiment, the light emitted from the LED 72 is distributed to the entire imaging visual field of the observation optical system 132. That is, the light distribution angle of the illumination unit 70 is equal to or greater than the viewing angle of the observation optical system 132. Therefore, in the endoscope 10 according to the fifth embodiment, it is possible to perform imaging with a sufficient light amount in the entire visual field of the observation optical system 132.

### REFERENCE SIGNS LIST

10 Endoscope
14 Insertion portion
13 Distal end portion
18 Channel outlet
131, 131A, 131B, 131C, 131D Distal end surface
132 Observation optical system
133 Recess
134, 135, 136 Protrusion
140 Air and water supply nozzle
141 Outlet

1. An endoscope that includes a convex observation optical system provided at a distal end of an insertion portion and in which a cleaning liquid is ejected from a nozzle, the endoscope comprising
a distal end surface surrounding the observation optical system and having an uneven shape.

2. The endoscope according to claim 1, wherein a plurality of recesses are provided on the distal end surface.

3. The endoscope according to claim 1, wherein a plurality of protrusions are provided on the distal end surface.

4. The endoscope according to claim 1, wherein the uneven shape forms a groove.

5. The endoscope according to claim 4, wherein the groove extends radially from the observation optical system.

6. The endoscope according to claim 4, wherein
a suction hole for sucking remaining liquid is formed on the distal end surface, and
the groove extends from the observation optical system toward the suction hole.

7. The endoscope according to claim 3, wherein each of the protrusions has a dot shape.

8. A method for manufacturing an endoscope that includes a convex observation optical system provided at a distal end of an insertion portion and in which a cleaning liquid is ejected from a nozzle, the method comprising
performing unevenness processing on a distal end surface surrounding the observation optical system.

9. A method for manufacturing an endoscope that includes a convex observation optical system provided at a distal end of an insertion portion and in which a cleaning liquid is ejected from a nozzle, the method comprising
forming a distal end surface surrounding the observation optical system and having an uneven shape by using a mold.
